(19) United States
(12) Patent Application Publication
(10) Pub. No.: US 2012/0239034 A1
(43) Pub. Date: Sep. 20, 2012

(54) Method of Manufacturing Tissue Seal Plates

(75) Inventors: Glenn A. Horner, Boulder, CO (US); Christina A. Oliver, Longmont, CO (US); Kim V. Brandt, Loveland, CO (US)

(73) Assignee: TYCO Healthcare Group LP, Boulder, CO (US)

(21) Appl. No.: 13/050,182

(22) Filed: Mar. 17, 2011

(51) Int. Cl. A61B 18/18

(52) U.S. Cl. 606/51

(57) ABSTRACT

An end effector assembly includes a pair of opposing jaw members, each jaw member having a jaw housing and a seal plate. The seal plate is associated with the jaw housing and includes an interior surface and an exterior surface. A portion of the exterior surface defines a tissue contacting surface and at least a portion of the interior surface includes a conductive element that is disposed thereon. The conductive element facilitates soldering a wire lead thereto for electrical communication with the seal plate.

### BACKGROUND

1. Technical Field

The present disclosure relates to electrosurgical instruments used for open and endoscopic surgical procedures. More particularly, the present disclosure relates to a method of manufacturing tissue seal plates for sealing vessels and vascular tissue.

2. Description of Related Art

Electrosurgical forceps utilize mechanical clamping action along with electrical energy to effect hemostasis on clamped tissue. The forceps (e.g., open, laparoscopic or endoscopic) include electrosurgical sealing plates that apply the electrosurgical energy to the clamped tissue. By controlling the intensity, frequency and duration of the electrosurgical energy applied through the seal plates to the tissue, the surgeon can coagulate, cauterize, and/or seal tissue therebetween.

Typically, an end effector assembly includes a pair of jaw members, each including a seal plate and an electrical lead. The seal plate is operably connected to an energy source, for example, an electrosurgical generator via the electrical lead. During a traditional manufacturing process, the wire lead is operably connected to a seal plate by crimping or welding via another coupling structure. These traditional fastening techniques are typically expensive and time consuming.

### SUMMARY

The present disclosure relates to an end effector assembly including a pair of opposing jaw members. Each jaw member has a jaw housing and a seal plate. The seal plate is associated within the jaw housing and includes an interior surface and an exterior surface. A portion of the exterior surface defines a tissue contacting surface and at least a portion of the interior surface includes a conductive element (e.g., gold and tin) that is disposed thereon. The conductive element facilitates soldering a wire lead thereto for electrical communication with the seal plate.

In embodiments, the conductive element is plated or clad onto the interior surface of the seal plate, for example, within a groove defined therein to facilitate engagement of the conductive element thereto. In other embodiments, the conductive element is coated onto the seal plate by an etching process.

The present disclosure also provides for a method of manufacturing an end effector assembly. The method includes the step of providing a seal plate. The method further includes the step of plating at least a portion of the seal plate with a conductive element. The seal plate may then be etched to remove at least a portion of the conductive element therefrom. In another step, an electrical lead is soldered to the conductive element.

In embodiments, before the etching step, an etch resist pattern may be applied to the seal plate.

### BRIEF DESCRIPTION OF THE DRAWINGS

Various embodiment of the subject instrument are described herein with reference to the drawings wherein:

FIG. 1A is a perspective view of an endoscopic forceps having an electrode assembly in accordance with an embodiment of the present disclosure;

FIG. 1B is a perspective view of an open forceps having an electrode assembly in accordance with an embodiment according to the present disclosure;

FIGS. 2A and 2B are exploded views of opposing jaw members of FIGS. 1A and 1B respectively;

FIG. 3A is a perspective view of opposing seal plates having a conductive element disposed thereon in accordance with an embodiment of the present disclosure;

FIG. 3B is a front, cross-sectional view of the opposing seal plates of FIG. 3A;

FIG. 3C is an enlarged view of a section of one of the opposing seal plates of FIG. 3B;

FIG. 4 is an enlarged view of one seal plate illustrating a conductive material within a groove in accordance with another embodiment of the present disclosure;

FIG. 5A is a perspective view of opposing seal plates having a conductive element disposed on a proximal face thereof, in accordance with another embodiment of the present disclosure;

FIG. 5B is a front, cross-sectional view of the opposing seal plates of FIG. 5A;

FIG. 6 is a flow-chart illustrating a method of manufacturing a seal plate, in accordance with an embodiment of the present disclosure; and

FIG. 7 is a flow-chart illustrating another method of manufacturing a seal plate, in accordance with another embodiment of the present disclosure.

### DETAILED DESCRIPTION

Embodiments of the presently-disclosed electrosurgical instrument are described in detail with reference to the drawings wherein like reference numerals identify similar or identical elements. As used herein, the term “distal” refers to a portion of an instrument or apparatus which is further from a user while the term “proximal” refers to a portion of the instrument or apparatus which is closer to a user.

In accordance with the present disclosure, an electrode assembly may be manufactured to include a conductive layer and/or coating such that an electrical lead may be operably connected (e.g., by soldering) to the electrode. In this manner, crimping and sonic welding connections are eliminated resulting in a less complicated and simpler electrical connecting technique.

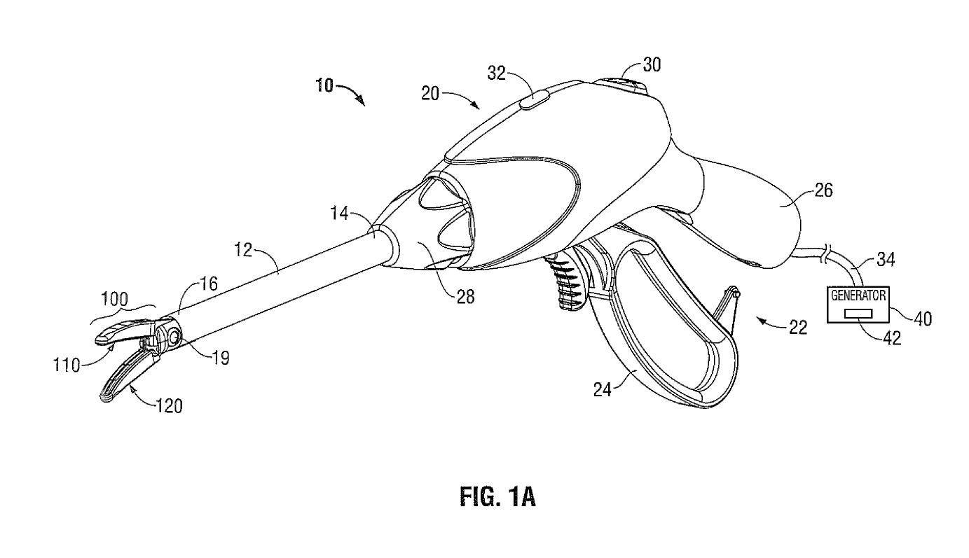

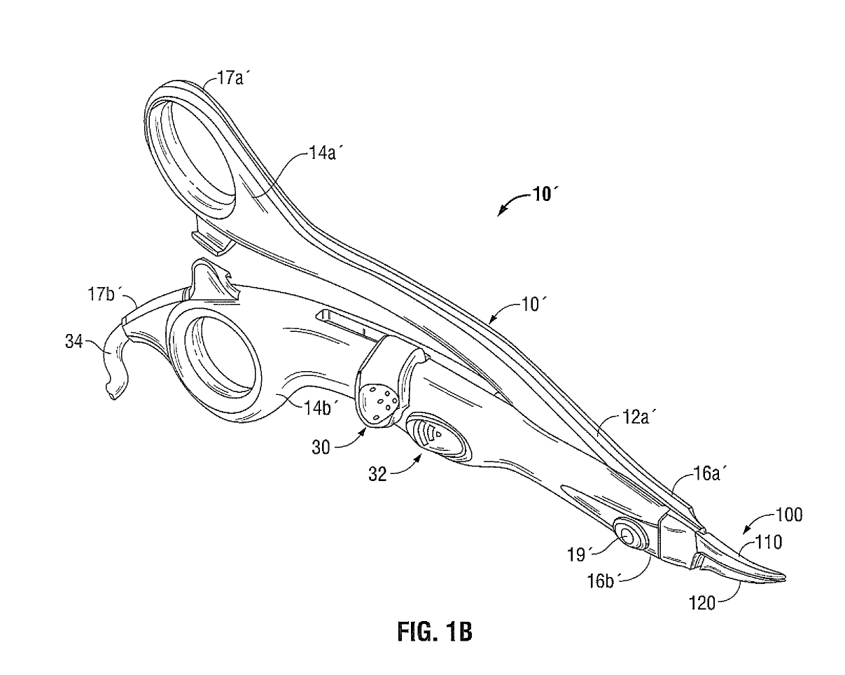

Referring now to the figures, FIG. 1A depicts an endoscopic forceps 10 as used in correlation with endoscopic surgical procedures and FIG. 1B depicts an open forceps 10′ as used in correlation with open surgical procedures. For the purposes herein, either an endoscopic instrument or an open surgery instrument may be utilized with the novel end effector assembly described herein. It should be noted that different electrical and mechanical connections and other considerations may apply to each particular type of instrument. However, the novel aspects, with respect to the end effector assembly and its operating characteristics, remain generally consistent with respect to both the endoscopic or open surgery designs.

The forceps 10 is coupled to a surgical energy source and adapted to seal tissue using radiofrequency (RF) energy. Surgical energy source (e.g., generator 40) is configured to output various types of energy such as RF energy having a frequency from about 300 MHz to about 5000 MHz. Forceps 10 is coupled to generator 40 via a cable 34 that is adapted to transmit the appropriate energy and control signals therebetween.

Forceps 10 is configured to support an end effector assembly 100 for sealing tissue. Forceps 10 typically includes various conventional features (e.g., a housing 20, a handle assembly 22, a rotating assembly 28, and a trigger assembly 30) that enable forceps 10 and end effector assembly 100 to mutually cooperate to grasp, seal, divide and/or sense tissue. Forceps 10 generally includes housing 20 and handle assembly 22, which includes a moveable handle 24 and a fixed handle 26 that is integral with housing 20. Handle 24 is moveable relative to fixed handle 26 to actuate end effector assembly 100 to grasp and treat tissue. Forceps 10 also includes a shaft 12 that has a distal portion 16 that mechanically engages end effector assembly 100 and a proximal portion 14 that mechanically engages housing 20 proximate the rotating assembly 28 disposed on housing 20. Rotating assembly 28 is mechanically associated with shaft 12 such that rotational movement of the rotating assembly 28 imparts similar rotational movement to shaft 12 which, in turn, rotates the end effector assembly 100.

End effector assembly 100 includes jaw members 110 and 120 where one or both are pivotable about a pin 19 from a first position wherein jaw members 110 and 120 are spaced relative to another, to a second position wherein jaw members 110 and 120 are closed and cooperate to grasp tissue therebetween.

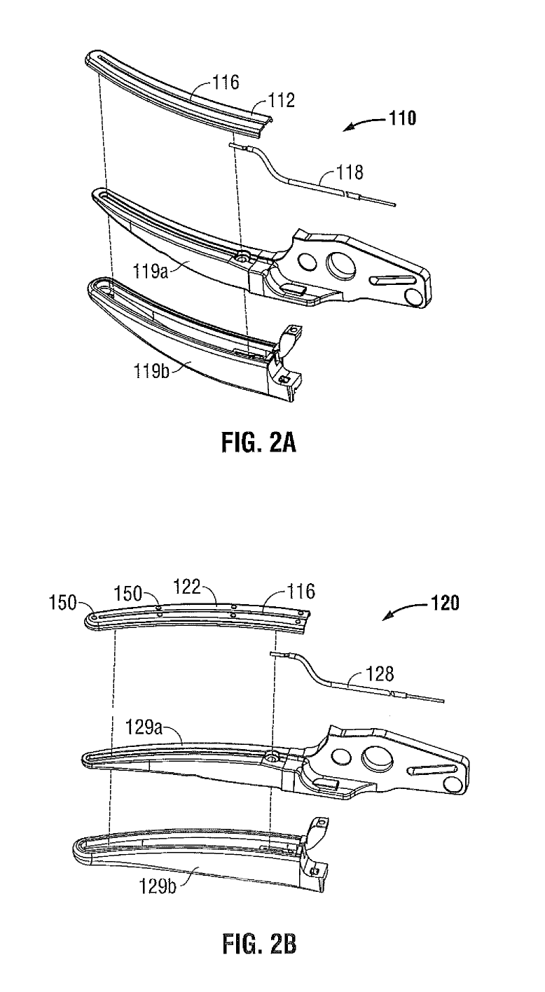

Each jaw member 110 and 120 includes a tissue contacting surface 112 and 122 (as shown in FIGS. 2A and 2B), respectively, disposed on an inner-facing surface thereof. Tissue contacting surfaces 112 and 122 cooperate to grasp, coagulate, seal, and/or cut tissue held therebetween upon selective application of energy from generator 40. Tissue contacting surfaces 112 and 122 are logically connected to generator 40, which, in turn, selectively communicates energy through the tissue held therebetween.

Trigger assembly 30 is configured to actuate a knife (not shown) disposed within forceps 10 to selectively sever tissue that is grasped between jaw members 110 and 120. A switch assembly 32 is configured to selectively provide electrosurgical energy to end effector assembly 100. Fixed handle 26 of handle assembly 22 is configured to receive a cable 34 that operably couples forceps 10 to generator 40.

Referring now to FIG. 1B, an open forceps 10′ is depicted and includes end effector assembly 100 (similar to forceps 10) that is attached to a handle assembly 22′ that includes a pair of elongated shaft portions 12a′ and 12b′. Each elongated shaft portion, 12a′ and 12b′, respectively, has a proximal end 14a′ and 14b′, respectively, and a distal end 16a′ and 16b′, respectively. The end effector assembly 100 includes jaw members 110 and 120 that attach to distal ends 16a′ and 16b′, respectively, of shafts 12a′ and 12b′, respectively. The jaw members 110 and 120 are connected about a pivot pin 19′ to allow jaw members 110 and 120 to pivot relative to one another from the first to second positions for treating tissue (as described above). Seal plates 112 and 122 (as shown in FIGS. 2A and 213) are connected to opposing jaw members 110 and 120 and include electrical leads 118, 128, respectively, through or around pivot pin 19′.

Each shaft 12a′ and 12b′ includes a handle 17a′ and 17b′ disposed at the proximal end 14a′ and 14b′ thereof. Handles 17a′ and 17b′ facilitate movement of the shafts 12a′ and 12b′ relative to one another which, in turn, pivot the jaw members 110 and 120 from the open position wherein the jaw members 110 and 120 are disposed in spaced relation relative to one another to the clamping or closed position wherein the jaw members 110 and 120 cooperate to grasp tissue therebetween.

In some embodiments, one or both of the shafts, e.g., shaft 12a′, includes a switch assembly 32′ that is configured to selectively provide electrical energy to seal plates 112 and 122 of the end effector assembly 100. Forceps 10′ is depicted having a cable 34′ that connects the forceps 10′ to generator 40 (as shown in FIG. 1A). Trigger assembly 30′ is configured to actuate a knife (not shown) disposed within forceps 10′ to selectively sever tissue that is grasped between jaw members 110 and 120.

FIGS. 2A and 2B are perspective views of opposing jaw members 110 and 120 according to one embodiment of the present disclosure. As mentioned above, each jaw member 110 and 120 includes a respective sealing plate 112, 122, a respective electrical jaw lead 118, 128, and a respective support base 119a, 129a. Support bases 119a and 129a are configured to support electrically conductive sealing plates 112 and 122 thereon. Sealing plates 112 and 122 may be affixed atop the support bases 119a and 129a, respectively, by any suitable method including but not limited to snap-fitting, overmolding, stamping, ultrasonic welding, etc. The support bases 119a and 129a and sealing plates 112 and 122 are at least partially encapsulated by insulative housings 119b and 129b, respectively, by way of an overmolding process to secure sealing plates 112 and 122 to support bases 119a and 129a, respectively. Electrical jaw lead 118 supplies a first electrical potential to sealing plate 112 and electrical jaw lead 128 supplies a second electrical potential to opposing sealing plate 122.

Jaw member 120 may also include a series of stop members 150 disposed on the inner facing surface of sealing plate 112 to facilitate gripping and manipulation of tissue and to define a gap between opposing jaw members 110 and 120 during sealing and cutting of tissue. The series of stop members 150 are applied onto the sealing plate 112 during manufacturing. Further, the sealing plates 112 and 122 include longitudinally-oriented knife slots 116 defined therethrough for reciprocation of a knife blade (not shown).

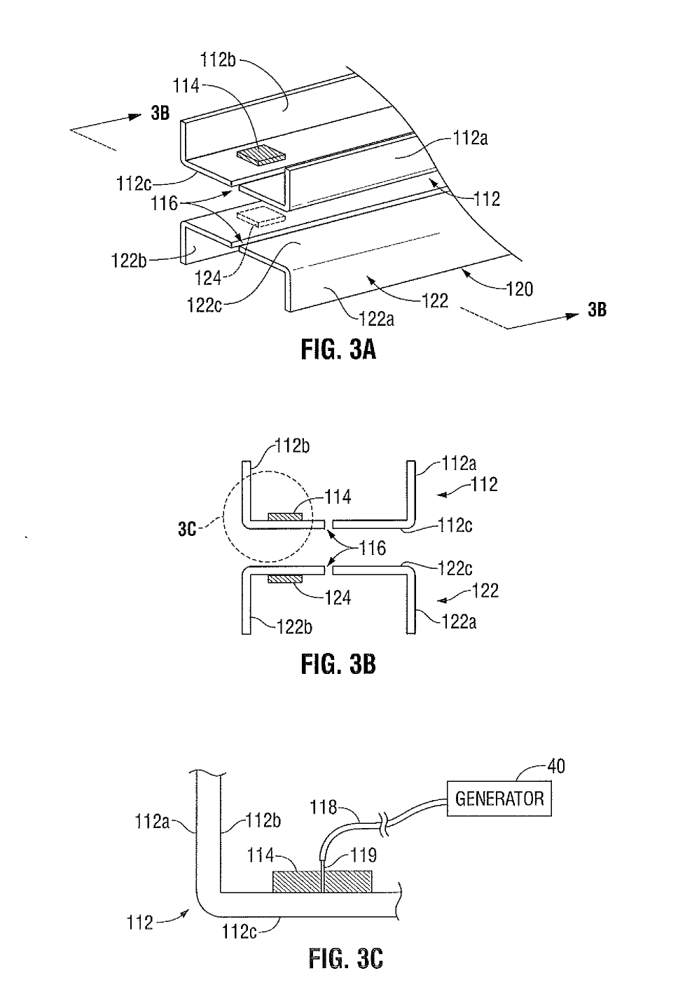

Referring now to FIGS. 3A-3C, each seal plate 112, 122 includes an outer portion 112a, 122a, respectively, and an inner portion 112b, 122b, respectively. Outer portion 112a, 122a provides the tissue contacting surface of respective seal plates 112 and 122 for grasping and sealing tissue therebetween. Inner portion 112b, 122b each include a conductive element 114, 124, respectively, applied thereon. Conductive elements 114, 124 facilitate a mechanical connection of respective wire leads 118 and 128 to inner portions 112b, 122b of seal plates 112 and 122. Each wire lead 118, 128 provides electrical communication between seal plates 112 and 122, respectively, and an energy source, control circuitry, or other electrical component(s). For example, wire leads 118, 128 may electrically couple seal plates 112, 122 to generator 40 (FIG. 1).

In FIG. 3C, seal plate 112 is shown having conductive element 114 disposed on inner portion 112b. Conductive element 114 may formed from gold, tin, or any other suitable material and may be disposed on inner portion 112b for this purpose by a suitable layer forming process, such as plating or cladding. As discussed above, wire lead 118 is operably coupled at one end to generator 40, for example, and on the other end to seal plate 112 via conductive element 114. More specifically, wire lead 118 is shown having a conductive segment 119 soldered to conductive element 114 so that electrical communication is provided between generator 40 and seal plate 112. The conductive element 114, e.g., gold or tin, facilitates the electrical connection between the wire lead 118 and the inner portion 112b of seal plate 112.

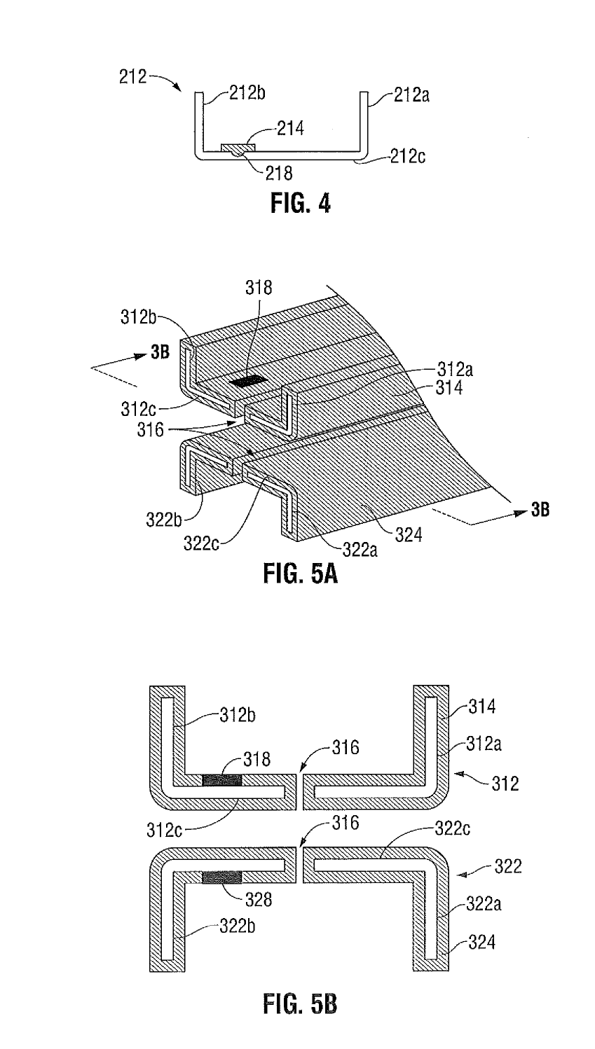

FIG. 4 illustrates another embodiment of a seal plate that is general depicted as 212. Similar to seal plate 112, seal plate 212 includes an outer portion 212a and an inner portion 212b. As described above, outer portion 212a provides a tissue contacting surface 212 that is configured to contact tissue held therebetween. Inner portion 212b provides a surface area for a conductive element 214 to be disposed thereon by cladding or plating or some other suitable process. Similar to seal plate 112, conductive element 214 facilitates coupling of a wire lead (e.g., wire lead 118 of FIG. 3C) to seal plate 212 since the wire lead may be soldered directly on the conductive element 214 on the seal plate 212. Inner portion 212b further includes a groove 218 defined therein to provide more surface area, thus providing a more secure connection between the conductive element 214 and seal plate 212. Groove 218 also provides a greater surface area for electrical communication between the wire lead and the inner portion 212b of seal plate 212 since a greater contact area is provided between conductive element 214 and seal plate 212 when the wire lead soldered to conductive element 214.

Referring now to FIGS. 5A and 5B, seal plates 312, 322 are shown and include an outer portion 312a, 322a and an inner portion 312b, 322b, similar to the seal plates described above. In this embodiment, a conductive material 314, 324 is formed on proximal face 312c, 322c of outer and inner portions 312a, 322a and 312b, 322b of seal plates 312 and 322, respectively. As will be described in greater detail below, a portion of the conductive material 314, 324 may then be removed from the respective seal plates 312 and 322, e.g., via etching, leaving the remaining portion 318, 328 of conductive material 314, 324, for coupling, e.g., soldering, of the wire leads (e.g., wire lead 118 (FIG. 3C)) thereto.

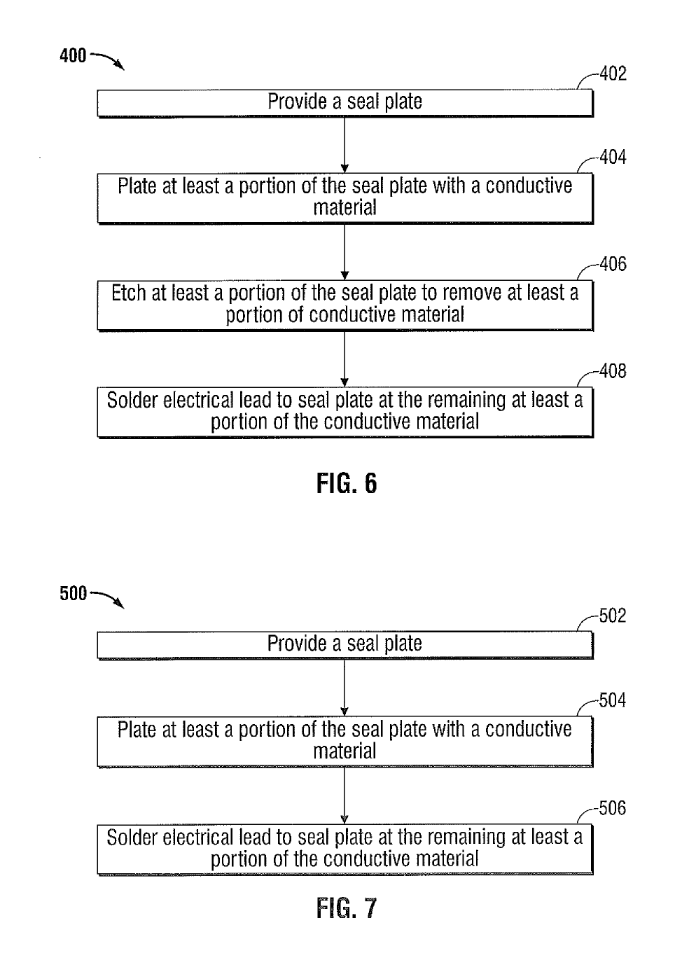

FIG. 6 illustrates a method 400 of manufacturing the novel end effector assembly in accordance with an embodiment of the present disclosure. In a first step 402, a seal plate 312, 322 is provided by a manufacturing process. In a next step 404, at least a portion of a surface of seal plate 312, 322 is coated with a conductive material 314, 324. The conductive material 314, 324 may be, for example, but not limited to gold or tin. All or a portion of the surface of seal plate 312, 322 may be plated/coated by any suitable method, for example, but not limited to electrolysis and cladding. In a next step 406, seal plate 312, 322 is etched so that a portion of unwanted conductive material is removed from seal plate 312, 322, leaving the remaining portion 318, 328 of conductive material 314, 324 disposed on seal plates 312, 322, respectively.

In another step, the etching is stopped when the desired amount of conductive material is left on seal plate 312, 322 that is needed for an appropriate solder, e.g., when remaining portions 318, 328 of conductive material 314, 324 is left. FIGS. 3A-3C show an example of a sealing plate having an appropriate or desired amount of conductive material disposed thereon. In an alternative step, when seal plate 312, 322 is completely or partially coated by conductive material 314, an etch resist pattern (not shown) may be applied thereto. In this manner, when the etching process is initiated only the portions where the etch resist pattern is exposed will be etched, thereby leaving a portion 318, 328 of desired conductive material 314 on an inner portion 312a, 322a of seal plates 312, 322, respectively.

In a step 408, a wire lead (e.g., 118) is soldered to the remaining portion of conductive material (see FIGS. 3A and 3B) to thereby provide electrical communication between seal plate 312, 322 and an electrosurgical energy source (e.g., generator 40). This procedure may be performed on one or both sealing plates 312 and 322.

FIG. 7 illustrates a method 500 of manufacturing the novel end effector assembly in accordance with an embodiment of the present disclosure. In step 502, a seal plate 312, 322 is provided in a manufacturing process. In step 504, at least a portion of seal plate 312, 322 is plated/coated with a conductive material 314, 324. In step 506, a wire lead (e.g., 118) is soldered to the remaining portion of conductive material (see FIGS. 3A and 3B) to thereby provide electrical communication between seal plate 312, 322 and an electrosurgical energy source (e.g., generator 40).

While several embodiments of the disclosure have been shown in the drawings and/or discussed herein, it is not intended that the disclosure be limited thereto, as it is intended that the disclosure be as broad in scope as the art will allow and that the specification be read likewise. For example, a conductive material may also be clad onto a sealing plate during a bending or stamping process during the manufacturing of the sealing plate. Therefore, the above description should not be construed as limiting, but merely as exemplifications of particular embodiments. Those skilled in the art will envision other modifications within the scope and spirit of the claims appended hereto.

What is claimed:

1. An end effector assembly, comprising:
a pair of opposing jaw members, each jaw member including:
a jaw housing; and
a seal plate associated with the jaw housing, the seal plate including an interior surface and an exterior surface, at least a portion of the exterior surface defining a tissue contacting surface, and at least a portion of the interior surface including a conductive element disposed thereon, wherein the conductive element facilitates soldering a wire lead thereto for electrical communication with the seal plate.

2. An end effector assembly according to claim 1, wherein the conductive element is formed from at least one of gold and tin.

3. An end effector assembly according to claim 1, wherein the conductive element is plated or clad onto the interior surface of the seal plate.

4. An end effector assembly according to claim 1, wherein the seal plate includes a groove defined therein to facilitate engagement of the conductive element thereto.

5. An end effector assembly according to claim 1, wherein the conductive element is coated onto the seal plate by an etching process.

6. A method of manufacturing an end effector assembly, the method comprising the steps of:
providing a seal plate having interior and exterior surfaces;
plating at least a portion of the interior surface of the seal plate with a conductive element;
etching at least a portion of the seal plate to remove at least a portion of the conductive element; and
soldering an electrical lead to the conductive element.

7. A method of manufacturing a seal plate according to claim 6, further comprising, before the etching step, applying an etch resist pattern to the seal plate.

8. A method of manufacturing an end effector assembly, the method comprising the steps of:
providing a seal plate having interior and exterior surfaces;
forming a groove on the interior surface;
plating at least a portion of the interior surface of the seal plate proximate the groove with a conductive material; and
soldering an electrical lead to the seal plate via the conductive material.
